(19) United States
(12) Patent Application Publication
(10) Pub. No.: US 2022/0139556 A1
(43) Pub. Date: May 5, 2022

(54) SYSTEM AND METHOD FOR DETERMINING PATIENT HEALTH INDICATORS THROUGH MACHINE LEARNING MODEL

(71) Applicant: Cloudphysician Healthcare Pvt Ltd, Banglore (IN)

(72) Inventors: Dileep C Unnikrishnan, Kerala (IN); Dileep Raman, Bengaluru (IN); Jitesh Sekar, Bengaluru (IN)

(73) Assignee: Cloudphysician Healthcare Pvt Ltd

(21) Appl. No.: 17/507,215

(22) Filed: Oct. 21, 2021

(30) Foreign Application Priority Data
Oct. 29, 2020 (IN) 202041047318

(51) Int. Cl. G16H 50/20; G16H 50/70; G16H 40/67; G16H 10/60; G16H 10/40; G16H 50/30; G16H 20/40; G06K 9/00; A61B 5/00; A61B 5/339

(52) U.S. Cl. CPC G16H 50/20; G16H 50/70; G16H 40/67; G16H 10/60; G16H 10/40; G16H 50/30; G16H 40/20; G06K 9/00664; A61B 5/7275; A61B 5/7267; A61B 5/339; A61B 5/4842; G16H 20/40

(57) ABSTRACT

This disclosure relates to method and system determining a plurality of patient health indicators through a Machine Learning (ML) model. The method includes receiving numerical data from a monitoring device. The numerical data is based on patient input data. The patient input data includes predefined variables, discretely sampled data, and continuously sampled data. The method further includes identifying a set of patterns from the numerical data through the ML model. The ML model is based on at least one of a Long Short Term Memory (LSTM), Extreme Gradient Boost (XGBoost), and Transformers. The method further includes comparing the set of patterns with historical medical data of the patient. The method further includes determining the plurality of patient health indicators through the ML model based on the comparing.

### DESCRIPTION

### Technical Field

This disclosure generally relates to determining patient health indicators and more particularly to method and system for determining patient health indicators through a Machine Learning (ML) model.

### Background

In the present age of automation, there is a need for innovation in automating healthcare infrastructure, particularly during pandemics such as novel Coronavirus Disease (nCOVID-19), to ensure preparedness and efficiency in patient management. In such times, as hospitals look for ways for increasing ventilator capacities, ensuring efficient management of each ventilator, it is essential to optimize ventilator usage. In present state of art, patient management is either manual or semi-automated. For example, for a patient on a ventilator, manual management requires staff members to frequently check on patient health and ventilator usage. Such methods are prone to observational errors and inaccurate monitoring. In case of semi-automated methods, certain key health parameters such as pulse rate, respiratory rate, and the like, are determined and monitored with predefined threshold values. However, frequent checks by staff members are still required to determine criticality of the patient.

Other conventional techniques for ventilator management and optimization include automated determination of criticality of patient health based on historical medical data of the patient. However, such techniques use a particular point of time for monitoring patient health through Intensive Care Unit (ICU) indices. Thus, such techniques fail to accurately identify patterns in the frequently monitored ICU indices.

In short, existing techniques fall short in providing a mechanism for monitoring health of patients on ventilator systems and other health monitoring systems and devices. Further, existing techniques fail to optimize usage and management of ventilator systems and other health monitoring devices.

### SUMMARY

In one embodiment, a method for determining a plurality of patient health indicators through a Machine Learning (ML) model is disclosed. In one example, the method may include receiving numerical data from a monitoring device. The numerical data is based on patient input data. The patient input data includes predefined variables, discretely sampled data, and continuously sampled data. The method further may include identifying a set of patterns from the numerical data through the ML model. The ML model is based on at least one of a Long Short Term Memory (LSTM), Extreme Gradient Boost (XGBoost), and Transformers. Further, the method may include comparing the set of patterns with historical medical data of the patient. Further, the method may include determining the plurality of patient health indicators through the ML model based on the comparing.

In one embodiment, a system for determining a plurality of patient health indicators through a Machine Learning (ML) model is disclosed. In one example, the system may include a processor and a computer-readable medium communicatively coupled to the processor. The computer-readable medium may store processor-executable instructions, which, on execution, may cause the processor to receive numerical data from a monitoring device. The numerical data is based on patient input data. The patient input data includes predefined variables, discretely sampled data, and continuously sampled data. The processor-executable instructions, on execution, may further cause the processor to identify a set of patterns from the numerical data through the ML model. The ML model is based on at least one of a Long Short Term Memory (LSTM), Extreme Gradient Boost (XGBoost), and Transformers. The processor-executable instructions, on execution, may further cause the processor to compare the set of patterns with historical medical data of the patient. The processor-executable instructions, on execution, may further cause the processor to determine the plurality of patient health indicators through the ML model based on the comparing.

In one embodiment, a non-transitory computer-readable medium storing computer-executable instructions for determining a plurality of patient health indicators through a Machine Learning (ML) model is disclosed. In one example, the stored instructions, when executed by a processor, may cause the processor to perform operations including receiving numerical data from a monitoring device. The numerical data is based on patient input data. The patient input data includes predefined variables, discretely sampled data, and continuously sampled data. The operations may further include identifying a set of patterns from the numerical data through the ML model. The ML model is based on at least one of a Long Short Term Memory (LSTM), Extreme Gradient Boost (XGBoost), and Transformers. The operations may further include comparing the set of patterns with historical medical data of the patient. The operations may further include determining the plurality of patient health indicators through the ML model based on the comparing.

It is to be understood that both the foregoing general description and the following detailed description are exemplary and explanatory only and are not restrictive of the invention, as claimed.

### BRIEF DESCRIPTION OF THE DRAWINGS

The accompanying drawings, which are incorporated in and constitute a part of this disclosure, illustrate exemplary embodiments and, together with the description, serve to explain the disclosed principles.

FIG. 1 is a block diagram of an exemplary system for determining a plurality of patient health indicators through a Machine Learning (ML) model, in accordance with some embodiments.

FIG. 2 is a functional block diagram of a health prediction device implemented by the exemplary system of FIG. 1, in accordance with some embodiments.

FIG. 3 is a flow diagram of an exemplary process for determining a plurality of patient health indicators through an ML model, in accordance with some embodiments.

FIG. 4 is a flow diagram of an exemplary process for transforming patient input data into numerical data, in accordance with some embodiments.

FIG. 5 illustrates training of an ML model based on Long Short Term Memory Recurrent Neural Network (LSTM RNN) algorithm, in accordance with some embodiments.

FIG. 6 illustrates training of an ML model based on Extreme Gradient Boost (XGBoost) algorithm, in accordance with some embodiments.

FIG. 7 is a block diagram of an exemplary computer system for implementing embodiments consistent with the present disclosure.

### DETAILED DESCRIPTION

Exemplary embodiments are described with reference to the accompanying drawings. Wherever convenient, the same reference numbers are used throughout the drawings to refer to the same or like parts. While examples and features of disclosed principles are described herein, modifications, adaptations, and other implementations are possible without departing from the spirit and scope of the disclosed embodiments. It is intended that the following detailed description be considered as exemplary only, with the true scope and spirit being indicated by the following claims.

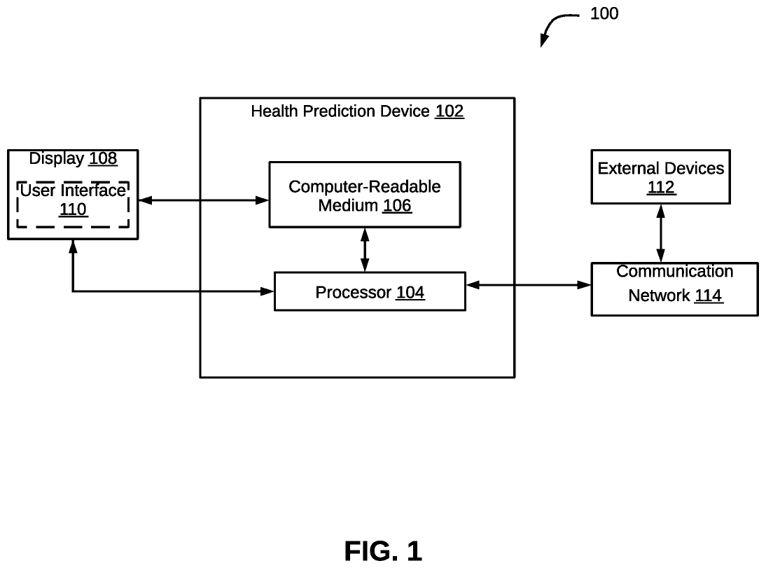

Referring now to FIG. 1, an exemplary system 100 for determining a plurality of patient health indicators through a Machine Learning (ML) model is illustrated, in accordance with some embodiments. The system 100 may implement in a health prediction engine, in accordance with some embodiments of the present disclosure. The health prediction engine may determine a plurality of patient health indicators through the ML model from numerical data corresponding to patient input data of a patient. In particular, the system 100 may include a health prediction device 102 (for example, server, desktop, laptop, notebook, netbook, tablet, smartphone, mobile phone, or any other computing device) that may implement the health prediction engine. It should be noted that, in some embodiments, the health prediction device 102 may determine a Deterioration Index (DI) and a clinical worsening score associated with the patient to analyze health of the patient.

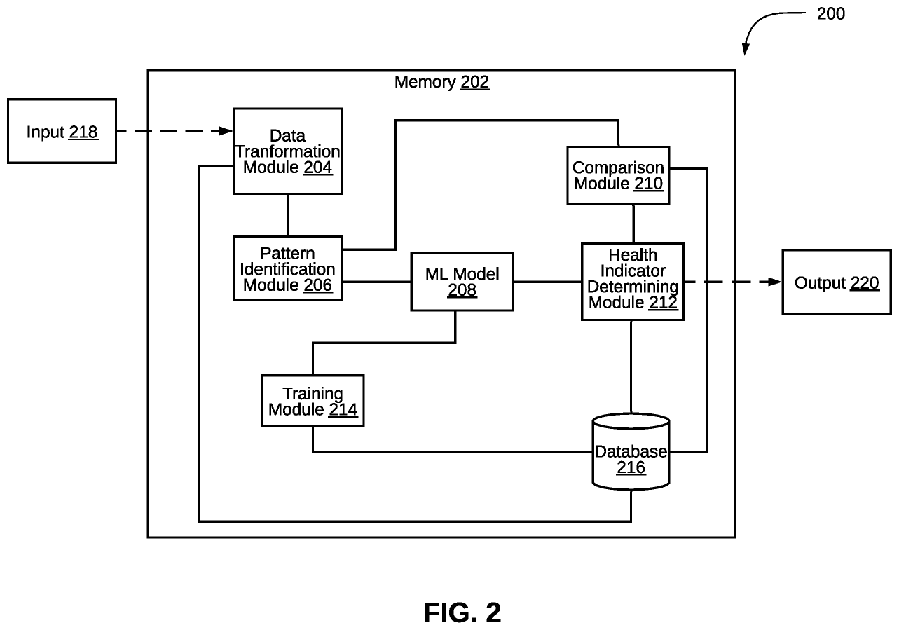

As will be described in greater detail in conjunction with FIGS. 2-6, the health prediction device 102 may receive numerical data from a monitoring device. Examples of the monitoring device, may include, but are not limited to ventilators and Electrocardiogram (ECG) monitor, Electroencephalogram (EEG) monitor, Electromyogram (EMG) monitor, and the like. The numerical data may be based on patient input data. It may be noted that the patient input data may include predefined variables, discretely sampled data, and continuously sampled data. By way of an example, the predefined variables may include, but may not be limited to, age, gender, pre-existing conditions, presenting symptoms, and the like. The discretely sampled data may include, but may not be limited to, key words in daily provider charts, laboratory data, intake output charts, hospital events, procedure details, administered drugs, key words in inter provider chat communication, and the like. The continuously sampled data may include, but may not be limited to, physiological, laboratory, and clinical parameters such as heart rate, respiratory rate, oxygen saturation, Glasgow Coma Scale, blood pressure, and the like. The health prediction device 102 may further identify a set of patterns from the numerical data through the ML model. The ML model may be based on at least one of a Long Short Term Memory (LSTM), Extreme Gradient Boost (XGBoost), and Transformers. The health prediction device 102 may further compare the set of patterns with historical medical data of the patient. The health prediction device 102 may further determine the plurality of patient health indicators through the ML model based on the comparing.

In some embodiments, the health prediction device 102 may include one or more processors 104 and a computer-readable medium 106 (for example, a memory). The computer-readable storage medium 106 may store instructions that, when executed by the one or more processors 104, cause the one or more processors 104 to determine a plurality of patient health indicators through the ML model, in accordance with aspects of the present disclosure. The computer-readable storage medium 106 may also store various data (for example, patient input data (such as a plurality of images and a plurality of videos associated with a ventilator display), numerical data based on the patient input data, training data, historical medical data, set of parameters for the ML model, and the like) that may be captured, processed, and/or required by the system 100.

The system 100 may further include a display 108. The system 100 may interact with a user via a user interface 110 accessible via the display 108. The system 100 may also include one or more external devices 112. In some embodiments, the health prediction device 102 may interact with the one or more external devices 112 over a communication network 114 for sending or receiving various data. The external devices 112 may include, but may not be limited to, a remote server, a digital device, or another computing system.

Referring now to FIG. 2, a functional block diagram of a health prediction device 200 is illustrated, in accordance with some embodiments. In particular, the health prediction device 200 may include, within a memory 202, a data transformation module 204, a pattern identification module 206, an ML model 208, a comparison module 210, a health indicator determining (HID) module 212, a training module 214, and a database 216. The memory 202 may receive an input 218 and provide an output 220. In some embodiments, the memory 202 may be analogous to the health prediction device 102 implemented by the system 100.

The data transformation module 204 may receive the input 218 from a monitoring device. The input 218 may include patient input data. It may be noted that the patient input data may include predefined variables, discretely sampled data, and continuously sampled data. By way of an example, the monitoring device may be a ventilator and the continuously sampled data may include a plurality of images and a plurality of videos corresponding to a display of the ventilator. Further, the data transformation module 204 may transform each of the patient input data into numerical data.

The discretely sampled data may be transformed into the numerical data through at least one Optical Character Recognition (OCR) technique and the continuously sampled data may be transformed into the numerical data through at least one Computer Vision (CV) technique. In continuation of the example above, the plurality of images and the plurality of videos may be obtained from the ventilator in real-time and the data transformation module 204 may transform each of the plurality of images and the plurality of videos into numerical data. The numerical data may be stored in the database 216.

Further, the numerical data may be sent from the data transformation module 204 to the pattern identification module 206. The pattern identification module 206 may identify a set of patterns from numerical data through the ML model 208. It may be noted that the ML model 208 is based on at least one of a Long Short Term Memory (LSTM), Extreme Gradient Boost (XGBoost), and Transformers. The ML model 208 may include a set of parameters. In an embodiment, an ML model based on LSTM may be based on Python 3.7 and Tensorflow 2.0. Further, the set of parameters may include a sequential model with one input layer of 32 nodes, a hidden layer of 32 nodes, an output layer of 1 node where the input layer and the hidden layer may be LSTM layers. In such an embodiment, the ML model may be compiled using binary cross entropy loss and rmsprop optimizer, and trained with a batch size of 64 for 20 epochs. In another embodiment, an ML model based on XGBoost may be based on Python 3.7, LightGBM (package version 2.3.2) and the set of parameters may include a depth of about 6, a learning rate of about 0.1, a number of leaves of about 31, about 85 iterations. Further, boosting for the ML model may be Gradient Boosting Decision Tree (GBDT), and loss for the ML model may be binary_crossentropy. In continuation of the example above, the pattern identification module 206 may identify the set of patterns from the numerical data based on the plurality of images and the plurality of videos corresponding to the ventilator display through the ML model 208.

Further, the comparison module 210 may receive the set of patterns from the pattern identification module 206. The comparison module 210 may compare the set of patterns with historical medical data of the patient to provide a comparison result. It may be noted that the historical medical data may be stored in the database 216. Further, the HID module 212 may receive the comparison result from the comparison module 210. The HID module 212 may determine the plurality of patient health indicators through the ML model 208 based on the comparison result. The plurality of patient health indicators may be stored in the database 216 as historical data associated with the patient, training data for the ML model 208, or the like. The HID module 212 may provide the plurality of patient health indicators as the output 220. By way of an example, the patient health indicators may include a clinical worsening probability score, a Deterioration Index (DI), a mortality probability score, a severity index, a criticality index, and a severity of illness score. Further, a treatment recommendation for the patient may be determined based on at least one of the plurality of patient health indicators. In an embodiment, the treatment recommendation may include, but may not be limited to, adding new medications, making adjustment in medication doses, procedure suggestions, and the like. Additionally, a plurality of parameters corresponding to the monitoring device may be determined based on at least one of the plurality of patient health indicators. In an embodiment, the HID module 212 may make recommendations to change a level of care associated with the patient (for example, transferring the patient to and from ICUs).

The HID module 212 may dynamically determine the DI associated with the patient based on the numerical data. Further, the HID module 212 may dynamically determine the clinical worsening probability score based on the set of patterns and the DI associated with the patient. In continuation of the example above, the HID module 212 may determine patient ventilator requirements through the ML model based on the numerical data corresponding to the patient. The patient ventilator requirements may include a ventilator stay, number of ventilator-free days, and a length of stay. The HID module 212 may dynamically determine the DI and the clinical worsening score associated with the patient using the ventilator. Additionally, the database 216 may include training data. The training module 214 may be used to train the ML model 208 based on the training data. By way of an example, the training data may include diagnostic data of the patient, historical medical data of the patient, Electronic Health Record (EHR) of the patient, Electronic Medical Record (EMR) of the patient, physician notes of the patient, laboratory data of the patient, and the like.

It should be noted that all such aforementioned modules 204-216 may be represented as a single module or a combination of different modules. Further, as will be appreciated by those skilled in the art, each of the modules 204-216 may reside, in whole or in parts, on one device or multiple devices in communication with each other. In some embodiments, each of the modules 204-216 may be implemented as dedicated hardware circuit comprising custom application-specific integrated circuit (ASIC) or gate arrays, off-the-shelf semiconductors such as logic chips, transistors, or other discrete components. Each of the modules 204-216 may also be implemented in a programmable hardware device such as a field programmable gate array (FPGA), programmable array logic, programmable logic device, and so forth. Alternatively, each of the modules 204-216 may be implemented in software for execution by various types of processors (e.g., processor 104). An identified module of executable code may, for instance, include one or more physical or logical blocks of computer instructions, which may, for instance, be organized as an object, procedure, function, or other construct. Nevertheless, the executables of an identified module or component need not be physically located together, but may include disparate instructions stored in different locations which, when joined logically together, include the module and achieve the stated purpose of the module. Indeed, a module of executable code could be a single instruction, or many instructions, and may even be distributed over several different code segments, among different applications, and across several memory devices.

As will be appreciated by one skilled in the art, a variety of processes may be employed for encrypting and decrypting a facial segment in an image with a unique server key. For example, the exemplary system 100 and the associated health prediction device 102, 200 may determine patient health indicators through an ML model by the processes discussed herein. In particular, as will be appreciated by those of ordinary skill in the art, control logic and/or automated routines for performing the techniques and steps described herein may be implemented by the system 100 and the associated health prediction device 102, 200 either by hardware, software, or combinations of hardware and software. For example, suitable code may be accessed and executed by the one or more processors on the system 100 to perform some or all of the techniques described herein. Similarly, application specific integrated circuits (ASICs) configured to perform some or all of the processes described herein may be included in the one or more processors on the system 100.

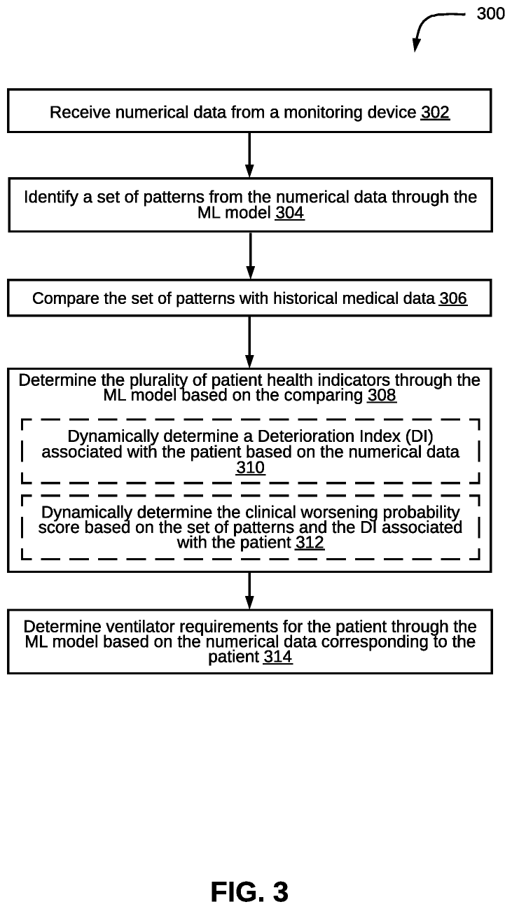

Referring to FIG. 3, an exemplary process 300 for determining a plurality of patient health indicators through an ML model (for example, the ML model 208) is illustrated via a flow chart, in accordance with some embodiments. The process 300 may be implemented by the health prediction device 102 of the system 100. The process 300 includes receiving numerical data from a monitoring device (for example, a ventilator), at step 302. It may be noted that the numerical data is based on patient input data. The patient input data includes predefined variables, discretely sampled data, and continuously sampled data. Further, the process 300 includes identifying a set of patterns from the numerical data through the ML model, at step 304. In some embodiments, the step 304 may be implemented by the pattern identification module 206 of the health prediction device 200. The ML model is based on at least one of an LSTM, an XGBoost, and Transformers. It may be noted that the ML model may be trained based on training data.

In some embodiments, the training may be performed by the training module 214 of the health prediction device 200. The training data may include diagnostic data of the patient, historical medical data of the patient, Electronic Health Record (EHR) of the patient, Electronic Medical Record (EMR) of the patient, physician notes of the patient, and laboratory data of the patient. Further, the process 300 includes comparing the set of patterns with historical medical data of the patient, at step 306. In some embodiments, the step 306 may be performed by the comparison module 210 of the health prediction device 200. Further, the process 300 includes determining the plurality of patient health indicators through the ML model based on the comparing, at step 308. By way of an example, the plurality of patient health indicators may include a clinical worsening probability score, a DI, a mortality probability score, a severity index, a criticality index, and a severity of illness score. Further, the step 308 includes dynamically determining the DI associated with the patient based on the numerical data, at step 310. It may be noted that the DI provides overall condition with respect to an event of interest (for example, intubation, seizure, cardiopulmonary arrest, death, or the like) of a patient by analyzing a plurality of tracked parameters over a hospital stay of the patient. Further, the step 308 includes dynamically determining the clinical worsening probability score based on the set of patterns and the DI associated with the patient, at step 312. In an embodiment, the monitoring device may be a ventilator. In such an embodiment, the continuously sampled data may include a plurality of images and a plurality of videos corresponding to a display of the ventilator. Further, the process 300 includes determining patient ventilator requirements for the patient through the ML model based on the numerical data corresponding to the patient, at step 314. The patient ventilator requirements include a ventilator stay, number of ventilator-free days, and a length of stay. In some embodiments, the steps 308-314 may be performed by the HID module 212 of the health prediction device 200. In another embodiment, the monitoring device may be a cardiac monitor. In such an embodiment, the continuously sampled data may include a plurality of images and a plurality of videos corresponding to a display of the cardiac monitor. The continuously sampled data may further include physiological data such as a heart rate of the patient, oxygen saturation of the patient, blood pressure of the patient, and the like.

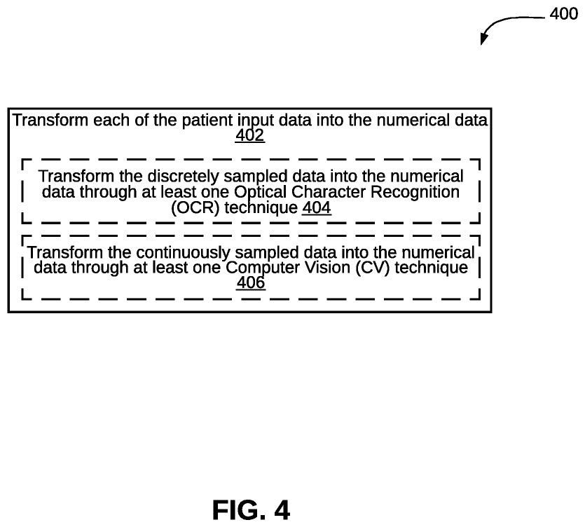

Referring to FIG. 4, an exemplary process 400 for transforming patient input data into numerical data is illustrated via a flow chart, in accordance with some embodiments. The process 400 may be implemented by the health prediction device 102 of the system 100. The process 400 includes transforming each of the patient input data into the numerical data, at step 402. Further, the process 400 includes transforming the discretely sampled data into the numerical data through at least one Optical Character Recognition (OCR) technique, at step 404. By way of an example, the at least one OCR technique may be based on, but not limited to, tesseract algorithm, Artificial Neural Network (ANN), Convolutional Neural Network (CNN), or a combination thereof. Further, the process 400 includes transforming the continuously sampled data into the numerical data through at least one Computer Vision (CV) technique, at step 406. By way of an example, the at least one CV technique may include, but may not be limited, CNN, region-based CNN, Recurrent Neural Network (RN N), semantic segmentation, or a combination thereof. The steps 402-406 of the process 400 may be implemented by the data transformation module 204 of the health prediction device 200.

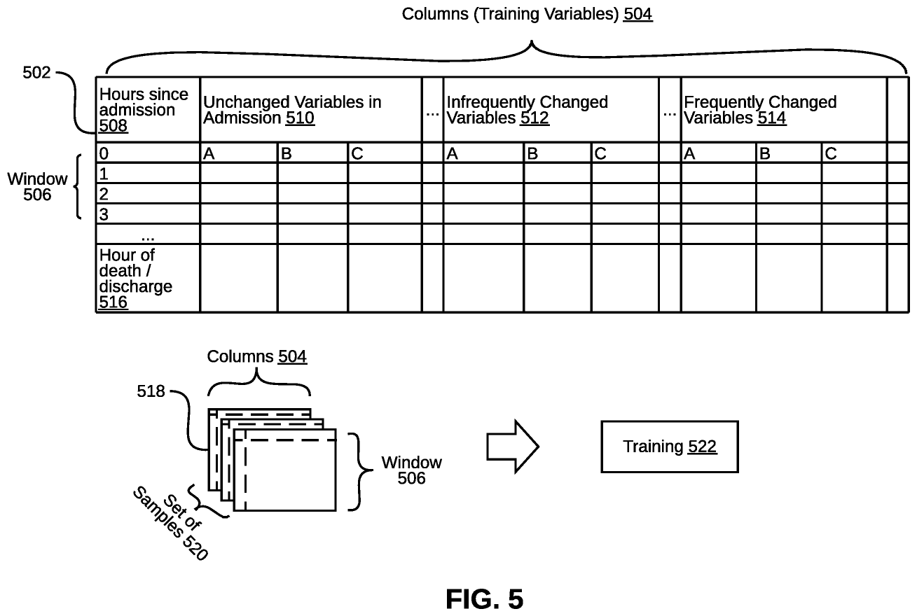

Referring now to FIG. 5, training of an ML model (for example, the ML model 208) based on LSTM RNN algorithm is illustrated, in accordance with some embodiments. The ML model may be trained based on training data. The training data may include diagnostic data of the patient, historical medical data of the patient, EHR of the patient, EMR of the patient, physician notes of the patient, and laboratory data of the patient. By way of an example, a table 502 represents a relational database including parameters of the EMR of the patient joined together. The table 502 includes columns 504 representing training variables of the EMR of the patient. In an embodiment, the training variables may be labelled as a hospital event of interest such as seizure, intubation, cardiopulmonary arrest, death, and the like. Further, the table 502 includes window 506 representing a particular time interval (for example, 1 hour, 2 hours, 3 hours, etc.). It may be noted that the window 506 for the EMR of the patient may extend up to an hour of death or discharge 516 of the patient.

Further, a training dataset 518 may be obtained from the table 502. A set of samples may be collected from the table 502 prior to an event of interest (for example, death) and an equal number of samples may be randomly generated to obtain a set of samples 520. In an exemplary scenario, a particular window of time “w” is selected prior to death of the patient. Further, a table including “w” number of rows and each of the columns 504 may be sampled from the table 502. It may be noted that the “w” rows sampled from the table 502 may be termed as positive labels. Further, “w” number of rows may be randomly generated. It may be noted that the randomly generated “w” rows may be termed as negative labels. The set of samples 520 is then used for training 522 the ML model through the training module 214.

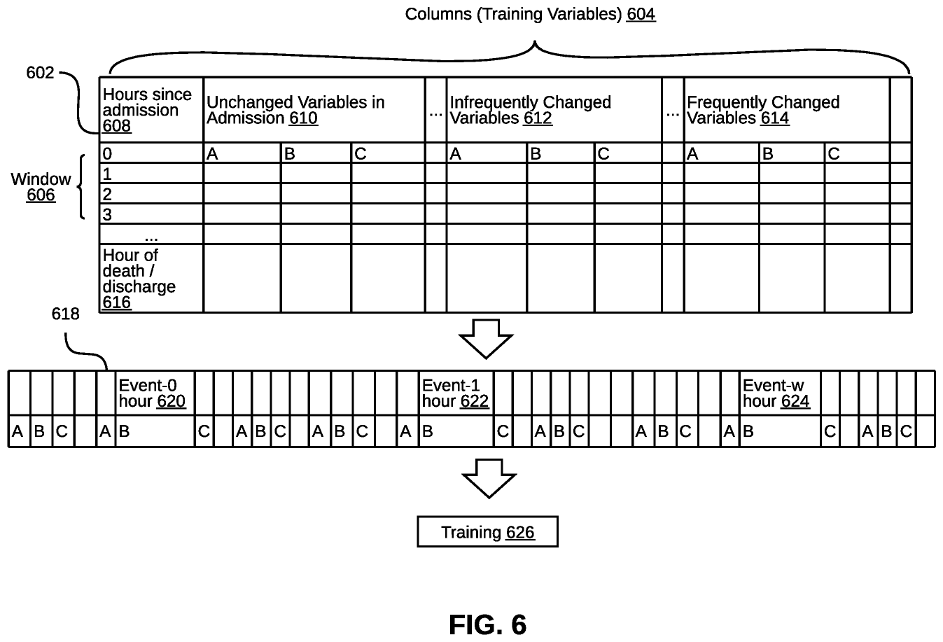

Referring now to FIG. 6, training of an ML model (for example, the ML model 208) based on XGBoost algorithm, in accordance with some embodiments is illustrated, in accordance with some embodiments. The ML model may be trained based on training data. The training data may include diagnostic data of the patient, historical medical data of the patient, EHR of the patient, EMR of the patient, physician notes of the patient, and laboratory data of the patient. By way of an example, a table 602 represents a relational database including parameters of the EMR of the patient joined together. The table 602 includes columns 604 representing training variables of the EMR of the patient. In an embodiment, the training variables may be labelled as a hospital event of interest such as seizure, intubation, cardiopulmonary arrest, death, and the like. Further, the table 602 includes window 606 representing a particular time interval (for example, 1 hour, 2 hours, 3 hours, etc.). It may be noted that the window 606 for the EMR of the patient may extend up to an hour of death or discharge 616 of the patient.

Further, a training vector 618 may be obtained from the table 602. It may be noted that the training vector 618 is a one-dimensional vector. The training vector 618 may be obtained by reshaping the table 602. In an exemplary scenario, a particular window of time “w” is selected prior to death of the patient. Further, a table including “w” number of rows and each of the columns 604 may be sampled from the table 602. Following reshaping, a one-dimensional vector may be obtained including each of the labels corresponding to the window 606 (for example, event-0 hour 620, event-1 hour 622, . . . , event-w hour 624, and the like). Further, the one-dimensional vector is then used for training 626 the ML model through the training module 214.

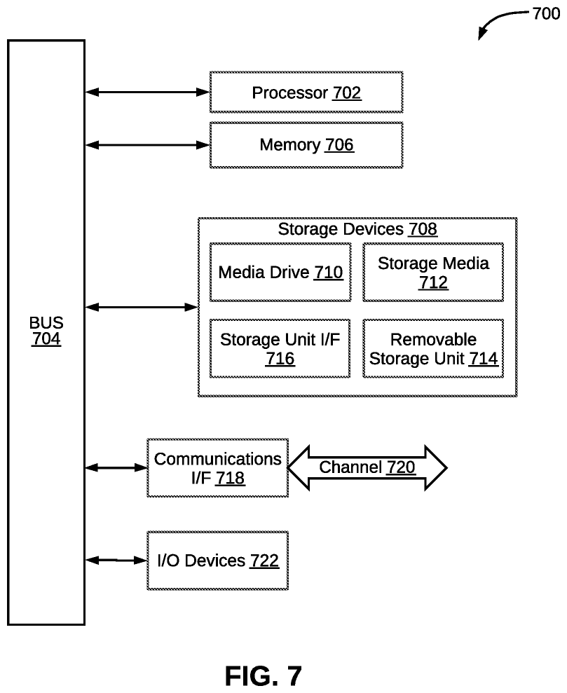

The disclosed methods and systems may be implemented on a conventional or a general-purpose computer system, such as a personal computer (PC) or server computer. Referring now to FIG. 7, an exemplary computing system 700 that may be employed to implement processing functionality for various embodiments (e.g., as a SIMD device, client device, server device, one or more processors, or the like) is illustrated. Those skilled in the relevant art will also recognize how to implement the invention using other computer systems or architectures. The computing system 700 may represent, for example, a user device such as a desktop, a laptop, a mobile phone, personal entertainment device, DVR, and so on, or any other type of special or general-purpose computing device as may be desirable or appropriate for a given application or environment. The computing system 700 may include one or more processors, such as a processor 702 that may be implemented using a general or special purpose processing engine such as, for example, a microprocessor, microcontroller or other control logic. In this example, the processor 702 is connected to a bus 704 or other communication medium. In some embodiments, the processor 702 may be an Artificial Intelligence (AI) processor, which may be implemented as a Tensor Processing Unit (TPU), or a graphical processor unit, or a custom programmable solution Field-Programmable Gate Array (FPGA).

The computing system 700 may also include a memory 706 (main memory), for example, Random Access Memory (RAM) or other dynamic memory, for storing information and instructions to be executed by the processor 702. The memory 706 also may be used for storing temporary variables or other intermediate information during execution of instructions to be executed by the processor 702. The computing system 700 may likewise include a read only memory (“ROM”) or other static storage device coupled to bus 704 for storing static information and instructions for the processor 702.

The computing system 700 may also include a storage devices 708, which may include, for example, a media drive 710 and a removable storage interface. The media drive 710 may include a drive or other mechanism to support fixed or removable storage media, such as a hard disk drive, a floppy disk drive, a magnetic tape drive, an SD card port, a USB port, a micro USB, an optical disk drive, a CD or DVD drive (R or RW), or other removable or fixed media drive. A storage media 712 may include, for example, a hard disk, magnetic tape, flash drive, or other fixed or removable medium that is read by and written to by the media drive 710. As these examples illustrate, the storage media 712 may include a computer-readable storage medium having stored therein particular computer software or data.

In alternative embodiments, the storage devices 708 may include other similar instrumentalities for allowing computer programs or other instructions or data to be loaded into the computing system 700. Such instrumentalities may include, for example, a removable storage unit 714 and a storage unit interface 716, such as a program cartridge and cartridge interface, a removable memory (for example, a flash memory or other removable memory module) and memory slot, and other removable storage units and interfaces that allow software and data to be transferred from the removable storage unit 714 to the computing system 700.

The computing system 700 may also include a communications interface 718. The communications interface 718 may be used to allow software and data to be transferred between the computing system 700 and external devices. Examples of the communications interface 718 may include a network interface (such as an Ethernet or other NIC card), a communications port (such as for example, a USB port, a micro USB port), Near field Communication (NFC), etc. Software and data transferred via the communications interface 718 are in the form of signals which may be electronic, electromagnetic, optical, or other signals capable of being received by the communications interface 718. These signals are provided to the communications interface 718 via a channel 720. The channel 720 may carry signals and may be implemented using a wireless medium, wire or cable, fiber optics, or other communications medium. Some examples of the channel 720 may include a phone line, a cellular phone link, an RF link, a Bluetooth link, a network interface, a local or wide area network, and other communications channels.

The computing system 700 may further include Input/Output (I/O) devices 722. Examples may include, but are not limited to a display, keypad, microphone, audio speakers, vibrating motor, LED lights, etc. The I/O devices 722 may receive input from a user and also display an output of the computation performed by the processor 702. In this document, the terms “computer program product” and “computer-readable medium” may be used generally to refer to media such as, for example, the memory 706, the storage devices 708, the removable storage unit 714, or signal(s) on the channel 720 . These and other forms of computer-readable media may be involved in providing one or more sequences of one or more instructions to the processor 702 for execution. Such instructions, generally referred to as “computer program code” (which may be grouped in the form of computer programs or other groupings), when executed, enable the computing system 700 to perform features or functions of embodiments of the present invention.

In an embodiment where the elements are implemented using software, the software may be stored in a computer-readable medium and loaded into the computing system 700 using, for example, the removable storage unit 714, the media drive 710 or the communications interface 718. The control logic (in this example, software instructions or computer program code), when executed by the processor 702, causes the processor 702 to perform the functions of the invention as described herein.

Thus, the disclosed method and system try to overcome the technical problem of determining a plurality of patient health indicators through a Machine Learning (ML) model. The method and system provide a high accuracy (discrimination and calibration) solution to determine health indicators and patient ventilator requirements. Image and video data from the ventilator is transformed into numerical data for efficient and less resource-intensive computation. The method and system further provide techniques for determining a Deterioration Index (DI) and clinical worsening score of the patient to analyze patient health and standardize management and classification of criticality of the patient.

As will be appreciated by those skilled in the art, the techniques described in the various embodiments discussed above are not routine, or conventional, or well understood in the art. The techniques discussed above provide for determining a plurality of patient health indicators through an ML model. The techniques first transform patient input data (for example, a plurality of images and a plurality of videos corresponding to a ventilator display) into numerical data. The techniques may then identify a set of patterns in the numerical data through the ML model. The techniques may then compare the set of patterns with historical medical data of the patient. The techniques may then determine the plurality of patient health indicators (for example, clinical worsening probability score, DI, mortality probability score, severity index, criticality index, severity of illness score, and the like) through the ML model based on the comparing.

In light of the above mentioned advantages and the technical advancements provided by the disclosed method and system, the claimed steps as discussed above are not routine, conventional, or well understood in the art, as the claimed steps enable the following solutions to the existing problems in conventional technologies. Further, the claimed steps clearly bring an improvement in the functioning of the device itself as the claimed steps provide a technical solution to a technical problem.

The specification has described method and system for determining a plurality of patient health indicators through a Machine Learning (ML) model. The illustrated steps are set out to explain the exemplary embodiments shown, and it should be anticipated that ongoing technological development will change the manner in which particular functions are performed. These examples are presented herein for purposes of illustration, and not limitation. Further, the boundaries of the functional building blocks have been arbitrarily defined herein for the convenience of the description. Alternative boundaries can be defined so long as the specified functions and relationships thereof are appropriately performed. Alternatives (including equivalents, extensions, variations, deviations, etc., of those described herein) will be apparent to persons skilled in the relevant art(s) based on the teachings contained herein. Such alternatives fall within the scope and spirit of the disclosed embodiments.

Furthermore, one or more computer-readable storage media may be utilized in implementing embodiments consistent with the present disclosure. A computer-readable storage medium refers to any type of physical memory on which information or data readable by a processor may be stored. Thus, a computer-readable storage medium may store instructions for execution by one or more processors, including instructions for causing the processor(s) to perform steps or stages consistent with the embodiments described herein. The term “computer-readable medium” should be understood to include tangible items and exclude carrier waves and transient signals, i.e., be non-transitory. Examples include random access memory (RAM), read-only memory (ROM), volatile memory, nonvolatile memory, hard drives, CD ROMs, DVDs, flash drives, disks, and any other known physical storage media.

It is intended that the disclosure and examples be considered as exemplary only, with a true scope and spirit of disclosed embodiments being indicated by the following claims.

What is claimed is:

1. A method for determining a plurality of patient health indicators through a Machine Learning (ML) model, the method comprising:
receiving, by a health prediction device, numerical data from a monitoring device, wherein the numerical data is based on patient input data, and wherein the patient input data comprises predefined variables, discretely sampled data, and continuously sampled data;
identifying, by the health prediction device, a set of patterns from the numerical data through the ML model, wherein the ML model is based on at least one of a Long Short Term Memory (LSTM), Extreme Gradient Boost (XGBoost), and Transformers;
comparing, by the health prediction device, the set of patterns with historical medical data of the patient; and
determining, by the health prediction device, the plurality of patient health indicators through the ML model based on the comparing.

2. The method of claim 1, further comprising training the ML model based on training data, wherein the training data comprises diagnostic data of the patient, historical medical data of the patient, Electronic Health Record (EHR) of the patient, Electronic Medical Record (EMR) of the patient, physician notes of the patient, and laboratory data of the patient.

3. The method of claim 1, further comprising transforming each of the patient input data into the numerical data.

4. The method of claim 3, wherein transforming each of the patient input data into the numerical data further comprises:
transforming the discretely sampled data into the numerical data through at least one Optical Character Recognition (OCR) technique; and
transforming the continuously sampled data into the numerical data through at least one Computer Vision (CV) technique.

5. The method of claim 1, wherein the plurality of patient health indicators comprises a clinical worsening probability score, a Deterioration Index (DI), a mortality probability score, a severity index, a criticality index, and a severity of illness score, wherein a treatment recommendation for the patient is determined based on at least one of the plurality of patient health indicators, and wherein a plurality of parameters corresponding to the monitoring device is determined based on at least one of the plurality of patient health indicators.

6. The method of claim 5, wherein determining the plurality of patient health indicators through the ML model further comprises:
dynamically determining the DI associated with the patient based on the numerical data; and
dynamically determining the clinical worsening probability score based on the set of patterns and the DI associated with the patient.

7. The method of claim 1, wherein the monitoring device is one of a ventilator or a cardiac monitor, and wherein the continuously sampled data comprises a plurality of images and a plurality of videos corresponding to a display of the one of the ventilator or the cardiac monitor.

8. The method of claim 7, further comprising determining patient ventilator requirements through the ML model based on the numerical data corresponding to the patient, wherein the patient ventilator requirements comprise a ventilator stay, number of ventilator-free days, and a length of stay.

9. A system for determining a plurality of patient health indicators through a Machine Learning (ML) model, the system comprising:
a processor; and
a memory communicatively coupled to the processor, wherein the memory stores processor instructions, which when executed by the processor, cause the processor to:
receive numerical data from a monitoring device, wherein the numerical data is based on patient input data, and wherein the patient input data comprises predefined variables, discretely sampled data, and continuously sampled data;
identify a set of patterns from the numerical data through the ML model, wherein the ML model is based on at least one of a Long Short Term Memory (LSTM), Extreme Gradient Boost (XGBoost), and Transformers;
compare the set of patterns with historical medical data of the patient; and
determine the plurality of patient health indicators through the ML model based on the comparing.

10. The system of claim 9, wherein the processor instructions, on execution, further cause the processor to train the ML model based on training data, wherein the training data comprises diagnostic data of the patient, historical medical data of the patient, Electronic Health Record (EHR) of the patient, Electronic Medical Record (EMR) of the patient, physician notes of the patient, and laboratory data of the patient.

11. The system of claim 9, wherein the processor instructions, on execution, further cause the processor to transform each of the patient input data into the numerical data.

12. The system of claim 11, wherein to transform each of the patient input data into the numerical data, the processor instructions, on execution, further cause the processor to:
transform the discretely sampled data into the numerical data through at least one Optical Character Recognition (OCR) technique; and
transform the continuously sampled data into the numerical data through at least one Computer Vision (CV) technique.

13. The system of claim 9, wherein the plurality of patient health indicators comprises a clinical worsening probability score, a Deterioration Index (DI), a mortality probability score, a severity index, a criticality index, and a severity of illness score, wherein a treatment recommendation for the patient is determined based on at least one of the plurality of patient health indicators, and wherein a plurality of parameters corresponding to the monitoring device is determined based on at least one of the plurality of patient health indicators.

14. The system of claim 13, wherein to determine the plurality of patient health indicators through the ML model, the processor instructions, on execution, further cause the processor to:
dynamically determine the DI associated with the patient based on the numerical data; and
dynamically determine the clinical worsening probability score based on the set of patterns and the DI associated with the patient.

15. The system of claim 9, wherein the monitoring device is one of a ventilator or a cardiac monitor, and wherein the continuously sampled data comprises a plurality of images and a plurality of videos corresponding to a display of the one of the ventilator or the cardiac monitor.

16. The system of claim 15, wherein the processor instructions, on execution, further cause the processor to determine patient ventilator requirements through the ML model based on the numerical data corresponding to the patient, wherein the patient ventilator requirements comprise a ventilator stay, number of ventilator-free days, and a length of stay.

17. A non-transitory computer-readable medium storing computer-executable instructions for determining a plurality of patient health indicators through a Machine Learning (ML) model, the computer-executable instructions configured for:
receiving numerical data from a monitoring device, wherein the numerical data is based on patient input data, and wherein the patient input data comprises predefined variables, discretely sampled data, and continuously sampled data;
identifying a set of patterns from the numerical data through the ML model, wherein the ML model is based on at least one of a Long Short Term Memory (LSTM), Extreme Gradient Boost (XGBoost), and Transformers;
comparing the set of patterns with historical medical data of the patient; and
determining the plurality of patient health indicators through the ML model based on the comparing.

18. The non-transitory computer-readable medium of claim 17, wherein the computer-executable instructions are further configured for transforming each of the patient input data into the numerical data.

19. The non-transitory computer-readable medium of claim 17, wherein the plurality of patient health indicators comprises a clinical worsening probability score, a Deterioration Index (DI), a mortality probability score, a severity index, a criticality index, and a severity of illness score, wherein a treatment recommendation for the patient is determined based on at least one of the plurality of patient health indicators, and wherein a plurality of parameters corresponding to the monitoring device is determined based on at least one of the plurality of patient health indicators.

20. The non-transitory computer-readable medium of claim 19, wherein for determining the plurality of patient health indicators through the ML model, the computer-executable instructions are further configured for:
dynamically determining the DI associated with the patient based on the numerical data; and
dynamically determining the clinical worsening probability score based on the set of patterns and the DI associated with the patient.
